(19) United States
(12) Patent Application Publication
(10) Pub. No.: US 2024/0407864 A1
(43) Pub. Date: Dec. 12, 2024

(54) LINEAR TRANSMISSION MECHANISM FOR ACTUATING A PRISMATIC JOINT OF A SURGICAL ROBOT

(71) Applicant: Covidien LP, Mansfield, MA (US)

(72) Inventor: Ulrich Hagn, Muenchen (DE)

(21) Appl. No.: 18/697,819

(22) PCT Filed: Nov. 7, 2022
(86) PCT No.: PCT/IB2022/060688

§ 371 Date: Apr. 2, 2024

Related U.S. Application Data
(60) Provisional application No. 63/276,726, filed on Nov. 8, 2021.

(51) Int. Cl. A61B 34/30

(52) U.S. Cl. CPC A61B 34/30

(57) ABSTRACT

A robotic arm with a linear transmission mechanism for a surgical robotic system. The robotic arm includes a first link pivotally coupled to a second link at a first joint and a holder pivotally coupled to the second link at a second joint.

### FIELD

The present disclosure generally relates to a surgical robotic system having a robotic arm with a linear transmission mechanism.

### BACKGROUND

Surgical robotic systems are currently being used in minimally invasive medical procedures. Some surgical robotic systems include a surgical console controlling a surgical robotic arm and a surgical instrument having an end effector (e.g., forceps or grasping instrument) coupled to and actuated by the robotic arm.

The surgical robotic arm may have multiple links and joints enabling versatile movements of the portions of the robotic arm and components coupled thereto.

### SUMMARY

According to one aspect of the present disclosure, a robotic arm for a surgical robotic system is disclosed and includes a first link, a second link, a holder, and an instrument drive unit. The first link is pivotally coupled to the second link at a first joint and the holder is pivotally coupled to the second link at a second joint. The first link includes an actuator and the second link includes a first belt operably coupled to the actuator. The holder includes a first pulley, a second pulley, and a second belt coupled to the first pulley and the second pulley. The instrument drive unit is operably coupled to the second belt and configured to move linearly along a longitudinal axis of the holder.

In an aspect, rotation of the actuator in a first direction causes the instrument drive unit to move along the longitudinal axis of the holder in a first linear direction and wherein rotation of the actuator in a second direction causes the instrument drive unit to move along the longitudinal axis of the holder in a second linear direction opposite the first linear direction.

In an aspect, the instrument drive unit is removably coupled to the second belt.

In an aspect, a surgical instrument is operably coupled to the instrument drive unit.

In an aspect, the actuator is integrated into the first link and is configured to produce an output oriented coaxially with the first joint.

In an aspect, the first pulley is coupled to the first belt at the second joint.

In an aspect, a third link is coupled to the first link at a third joint.

In an aspect, a second actuator is disposed at the third joint. Additionally or alternatively, the first link further includes a third belt operably coupled to the second actuator. In an aspect, actuation of the second actuator causes the first link to pivot relative to the third link and the second link.

In an aspect, angle between the holder and the first link does not change upon movement of the second link relative to the first link.

According to another aspect of the present disclosure, a robotic surgical system is disclosed and includes a movable cart and a robotic arm operably coupled to the movable cart. The robotic arm includes a first link, a second link, a holder, and an instrument drive unit. The first link is pivotally coupled to the second link at a first joint and the holder is pivotally coupled to the second link at a second joint. The first link includes an actuator and the second link includes a first belt operably coupled to the actuator. The holder includes a first pulley, a second pulley, and a second belt coupled to the first pulley and the second pulley. The instrument drive unit is operably coupled to the second belt and configured to move linearly along a longitudinal axis of the holder.

In an aspect, rotation of the actuator in a first direction causes the instrument drive unit to move along the longitudinal axis of the holder in a first linear direction and wherein rotation of the actuator in a second direction causes the instrument drive unit to move along the longitudinal axis of the holder in a second linear direction opposite the first linear direction.

In an aspect, the instrument drive unit is removably coupled to the second belt.

In an aspect, a surgical instrument is operably coupled to the instrument drive unit.

In an aspect, the actuator is integrated into the first link and is configured to produce an output oriented coaxially with the first joint.

In an aspect, the first pulley is coupled to the first belt at the second joint.

In an aspect, a third link is coupled to the first link at a third joint.

In an aspect, a second actuator is disposed at the third joint. Additionally or alternatively, the first link further includes a third belt operably coupled to the second actuator. In an aspect, actuation of the second actuator causes the first link to pivot relative to the third link and the second link.

In an aspect, angle between the holder and the first link does not change upon movement of the second link relative to the first link.

### BRIEF DESCRIPTION OF THE DRAWINGS

Various embodiments of the present disclosure are described herein with reference to the drawings, wherein:

FIG. 1 is a schematic illustration of a surgical robotic system including a control tower, a console, and one or more surgical robotic arms according to an embodiment of the present disclosure;

FIG. 2 is a perspective view of a surgical robotic arm of the surgical robotic system of FIG. 1 according to an embodiment of the present disclosure;

FIG. 3 is a perspective view of a setup arm with the surgical robotic arm of the surgical robotic system of FIG. 1 according to an embodiment of the present disclosure;

FIG. 4 is a schematic diagram of a computer architecture of the surgical robotic system of FIG. 1 according to an embodiment of the present disclosure; and

FIG. 5 is a perspective view of the surgical robotic arm of FIG. 2 schematically illustrating internal components thereof depicted externally for visibility.

### DETAILED DESCRIPTION

The term “application” may include a computer program designed to perform functions, tasks, or activities for the benefit of a user. Application may refer to, for example, software running locally or remotely, as a standalone program or in a web browser, or other software which would be understood by one skilled in the art to be an application. An application may run on a controller, or on a user device, including, for example, a mobile device, an IoT device, or a server system.

As will be described in detail below, the present disclosure is directed to a surgical robotic system, which includes a surgical console, a control tower, and one or more movable carts having a surgical robotic arm coupled to a setup arm. The surgical console receives user input through one or more interface devices, which are interpreted by the control tower as movement commands for moving the surgical robotic arm. The surgical robotic arm includes a controller, which is configured to process the movement command and to generate a torque command for activating one or more actuators of the robotic arm, which would, in turn, move the robotic arm in response to the movement command.

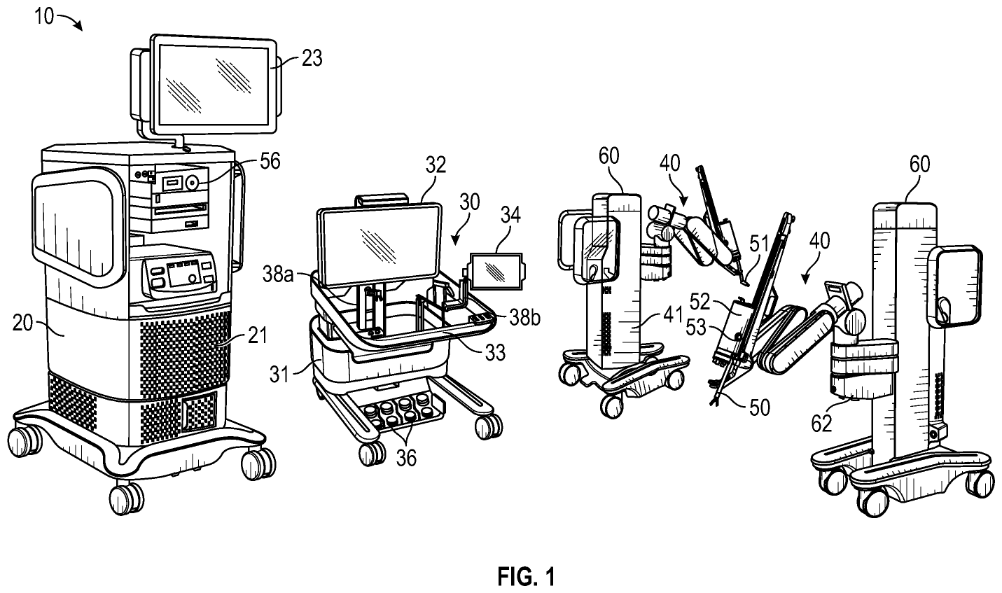

With reference to FIG. 1, a surgical robotic system 10 includes a control tower 20, which is connected to all of the components of the surgical robotic system 10 including a surgical console 30 and one or more robotic arms 40. Each of the robotic arms 40 includes a surgical instrument 50 removably coupled thereto. Each of the robotic arms 40 is also coupled to a movable cart 60.

The surgical instrument 50 is configured for use during minimally invasive surgical procedures. In embodiments, the surgical instrument 50 may be configured for open surgical procedures. In embodiments, the surgical instrument 50 may be an endoscope configured to provide a video feed for the user. In further embodiments, the surgical instrument 50 may be an electrosurgical forceps configured to seal tissue by compression tissue between jaw members and applying electrosurgical current thereto. In yet further embodiments, the surgical instrument 50 may be a surgical stapler including a pair of jaws configured to grasp and clamp tissue whilst deploying a plurality of tissue fasteners, e.g., staples, and cutting stapled tissue.

Each of the robotic arms 40 may include a camera 51 configured to capture video of the surgical site. The camera 51 may be a stereoscopic camera and may be disposed along with the surgical instrument 50 on the robotic arm 40. The surgical console 30 includes a first display 32, which displays a video feed of the surgical site provided by camera 51 of the surgical instrument 50 disposed on the robotic arms 40, and a second display device 34, which displays a user interface for controlling the surgical robotic system 10. The surgical console 30 also includes a plurality of user interface devices, such as foot pedals 36 and a pair of handle controllers 38a and 38b which are used by a user to remotely control robotic arms 40.

The control tower 20 includes a display 23, which may be a touchscreen, and outputs on the graphical user interfaces (GUIs). The control tower 20 also acts as an interface between the surgical console 30 and one or more robotic arms 40. In particular, the control tower 20 is configured to control the robotic arms 40, such as to move the robotic arms 40 and the corresponding surgical instrument 50, based on a set of programmable instructions and/or input commands from the surgical console 30, in such a way that robotic arms 40 and the surgical instrument 50 execute a desired movement sequence in response to input from the foot pedals 36 and the handle controllers 38a and 38b.

Each of the control tower 20, the surgical console 30, and the robotic arm 40 includes a respective computer 21, 31, 41. The computers 21, 31, 41 are interconnected to each other using any suitable communication network based on wired or wireless communication protocols. The term “network,” whether plural or singular, as used herein, denotes a data network, including, but not limited to, the Internet, Intranet, a wide area network, or a local area networks, and without limitation as to the full scope of the definition of communication networks as encompassed by the present disclosure. Suitable protocols include, but are not limited to, transmission control protocol/internet protocol (TCP/IP), datagram protocol/internet protocol (UDP/IP), and/or datagram congestion control protocol (DCCP). Wireless communication may be achieved via one or more wireless configurations, e.g., radio frequency, optical, Wi-Fi, Bluetooth (an open wireless protocol for exchanging data over short distances, using short length radio waves, from fixed and mobile devices, creating personal area networks (PANs), ZigBee® (a specification for a suite of high level communication protocols using small, low-power digital radios based on the IEEE 802.15.4-2003 standard for wireless personal area networks (WPANs)).

The computers 21, 31, 41 may include any suitable processor (not shown) operably connected to a memory (not shown), which may include one or more of volatile, non-volatile, magnetic, optical, or electrical media, such as read-only memory (ROM), random access memory (RAM), electrically-erasable programmable ROM (EEPROM), non-volatile RAM (NVRAM), or flash memory. The processor may be any suitable processor (e.g., control circuit) adapted to perform the operations, calculations, and/or set of instructions described in the present disclosure including, but not limited to, a hardware processor, a field programmable gate array (FPGA), a digital signal processor (DSP), a central processing unit (CPU), a microprocessor, and combinations thereof. Those skilled in the art will appreciate that the processor may be substituted for by using any logic processor (e.g., control circuit) adapted to execute algorithms, calculations, and/or set of instructions described herein.

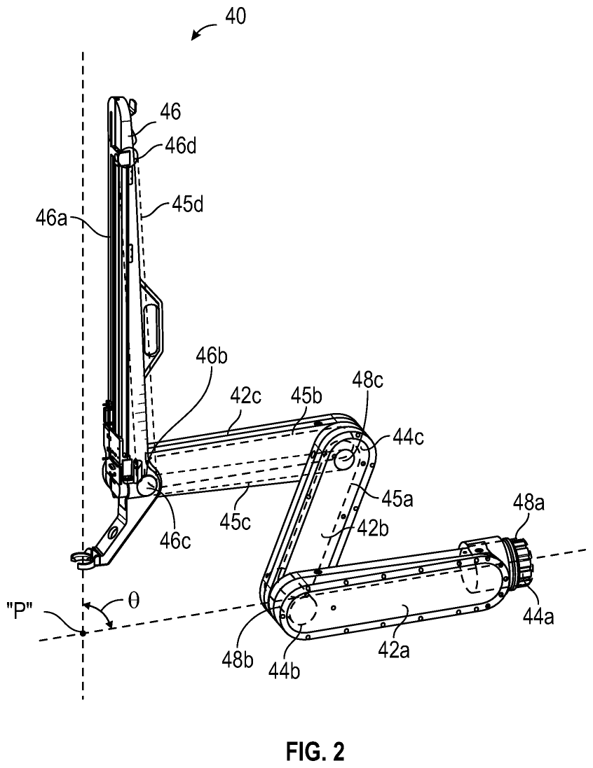

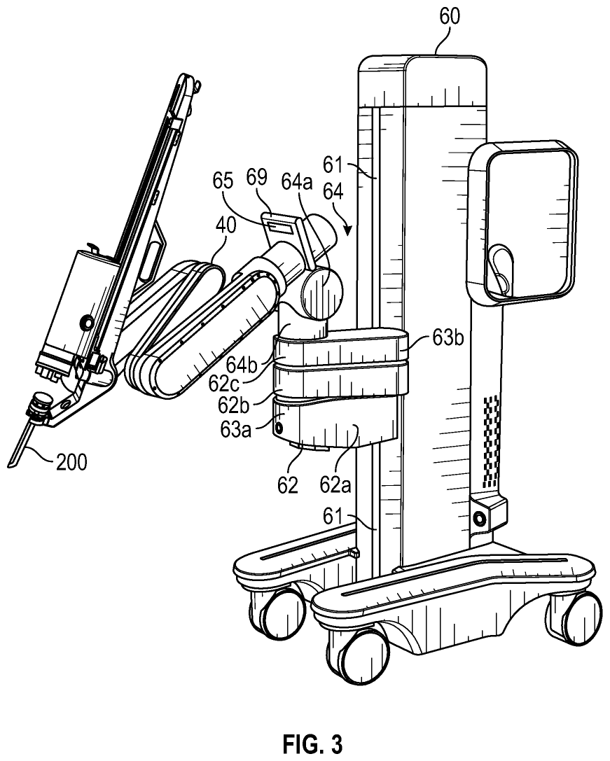

With reference to FIG. 2, each of the robotic arms 40 may include a plurality of links 42a, 42b, 42c, which are interconnected at joints 44a, 44b, 44c, respectively. The joint 44a is configured to secure the robotic arm 40 to the movable cart 60 and defines a first longitudinal axis. With reference to FIG. 3, the movable cart 60 includes a lift 61 and a setup arm 62, which provides a base for mounting of the robotic arm 40. The lift 61 allows for vertical movement of the setup arm 62. The movable cart 60 also includes a display 69 for displaying information pertaining to the robotic arm 40.

The setup arm 62 includes a first link 62a, a second link 62b, and a third link 62c, which provide for lateral maneuverability of the robotic arm 40. The links 62a, 62b, 62c are interconnected at joints 63a and 63b, each of which may include an actuator (not shown) for rotating the links 62b and 62b relative to each other and the link 62c. In particular, the links 62a, 62b, 62c are movable in their corresponding lateral planes that are parallel to each other, thereby allowing for extension of the robotic arm 40 relative to the patient (e.g., surgical table). In embodiments, the robotic arm 40 may be coupled to the surgical table (not shown). The setup arm 62 includes controls 65 for adjusting movement of the links 62a, 62b, 62c as well as the lift 61.

The third link 62c includes a rotatable base 64 having two degrees of freedom. In particular, the rotatable base 64 includes a first actuator 64a and a second actuator 64b. The first actuator 64a is rotatable about a first stationary arm axis which is perpendicular to a plane defined by the third link 62c and the second actuator 64b is rotatable about a second stationary arm axis which is transverse to the first stationary arm axis. The first and second actuators 64a and 64b allow for full three-dimensional orientation of the robotic arm 40.

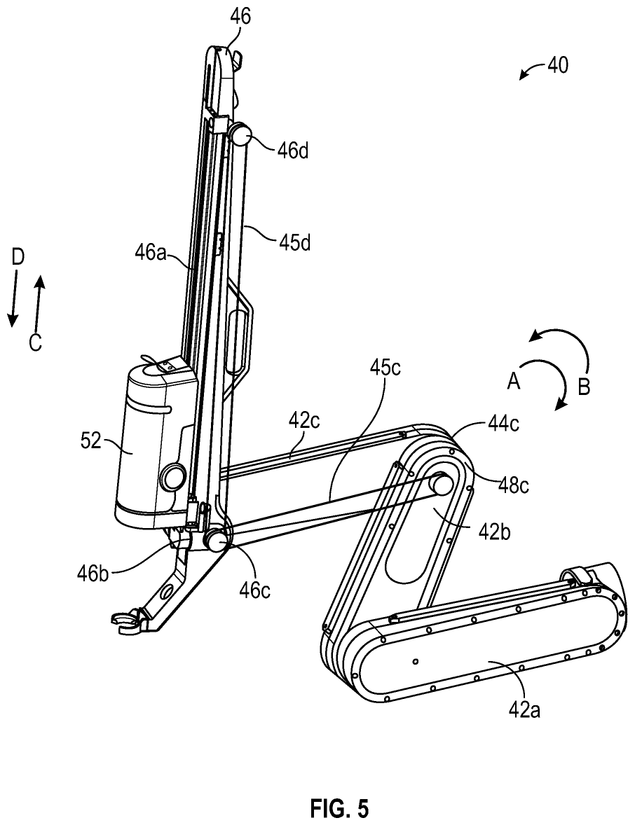

With reference to FIG. 2, the robotic arm 40 also includes a holder 46 defining a second longitudinal axis and configured to receive the instrument drive unit 52 (FIG. 1) of the surgical instrument 50, which is configured to couple to an actuation mechanism of the surgical instrument 50. Instrument drive unit 52 transfers actuation forces from its actuators to the surgical instrument 50 to actuate components (e.g., end effectors) of the surgical instrument 50. The holder 46 includes a sliding mechanism 46a, which is configured to move the instrument drive unit 52 along the second longitudinal axis defined by the holder 46 in the direction of arrow “C” and the direction of arrow “D” (FIG. 5). The holder 46 also includes a joint 46b, which rotates the holder 46 relative to the link 42c.

The robotic arm 40 also includes a plurality of manual override buttons 53 disposed on the IDU 52 and the setup arm 62, which may be used in a manual mode. The clinician may press one or the buttons 53 to move the component associated with the button 53.

The joints 44a and 44b include an actuator 48a and 48b configured to drive the joints 44a, 44b, 44c relative to each other through a series of belts 45a and 45b or other mechanical linkages such as a drive rod, a cable, or a lever and the like. In particular, the actuator 48a is configured to rotate the robotic arm 40 about a longitudinal axis defined by the link 42a.

The actuator 48b of the joint 44b is coupled to the joint 44c via the belt 45a, and the joint 44c is in turn coupled to the joint 46b via the belt 45b. Joint 44c may include a transfer case coupling the belts 45a and 45b, such that the actuator 48b is configured to rotate each of the links 42b, 42c and the holder 46 relative to each other. More specifically, links 42b, 42c, and the holder 46 are passively coupled to the actuator 48b which enforces rotation about a pivot point “P” which lies at an intersection of the first axis defined by the link 42a and the second axis defined by the holder 46. Thus, the actuator 48b controls the angle θ between the first and second axes allowing for orientation of the surgical instrument 50. Due to the interlinking of the links 42a, 42b, 42c, and the holder 46 via the belts 45a and 45b, the angles between the links 42a, 42b, 42c, and the holder 46 are also adjusted in order to achieve the desired angle θ. In embodiments, some or all of the joints 44a, 44b, 44c may include an actuator to obviate the need for mechanical linkages.

With continued reference to FIG. 2 and with additional reference to FIG. 5, linear movement of the instrument drive unit 52 along the second longitudinal axis defined by the holder 46 in the direction of arrow “C” and the direction of arrow “D”, and the components causing such movements, will now be described. As described above, the robotic arm 40 includes a first link 42b pivotably coupled to a second link 42c and a holder 46 pivotably coupled to the second link 42c. In an aspect, the robotic arm 40 includes one or more additional links, such as third link 42a. The first link 42b is coupled to the second link 42c at the first joint 44c and the holder 46 is coupled to the second link 42c at the second joint 46b. The third link 42a is coupled to the first link 42b at the third joint 44b.

The second link 42c includes a first belt 45c. The first link 42b integrates an actuator 48c, with its rotary output being coaxial to the first joint 44c and operably coupled to the first belt 45c. The holder 46 includes a first pulley 46c, a second pulley 46d, and a second belt 45d coupled to the first pulley 46c and the second pulley 46d. The first belt 45c is also operationally coupled to the first pulley 46c. The instrument drive unit 52 is operably coupled to the second belt 45d and is detachable therefrom. The instrument drive unit 52 is configured to move linearly along a longitudinal axis of the holder 46 (e.g., second longitudinal axis described above) and detachable couples a surgical instrument 50 to the robotic arm 40.

Rotation of the actuator 48c in a first direction “A” causes the instrument drive unit 52 to move along the longitudinal axis of the holder 46 in a first linear direction “C” and rotation of the actuator 48c in a second direction “B”, opposite the first direction “A”, causes the instrument drive unit 52 to move along the longitudinal axis of the holder 46 in a second linear direction “D” opposite the first linear direction “C”.

The actuator 48c may be mounted into the first link 42b and its output is positioned at the first joint 44c and the first pulley 46c is positioned at the second joint 46b. The first pulley 46c is coupled to the actuator 48c via the first belt 45c of the second link 42c. A second actuator 48b is positioned at the third joint 44b and is coupled to a third belt 45a, which is included in the first link 42b, and to a fourth belt 45b, which is included in the second link 42c, such that actuation of the second actuator 48b causes the first link 42b to pivot relative to the third link 42a and the second link 42c. In other words, this movement mechanism consist of the second actuator 48b (mounted to the third link 42a) and two drive mechanisms (e.g., cables, belts) of which the first (e.g., third belt 45a) connects the third link 42a with the second link 42c and the second (e.g., fourth belt 45b) connects the first link 42b with the holder 46.

As the first link 42b pivots relative to the third link 42a, the angle between the holder 46 and the third link 42a and the angle between the holder 46 and the second link 42c remains unchanged. Additionally, according to the disclosed configuration of the robotic arm 40, as any of the first link 42b, second link 42c, third link 42a, and the holder 46 are pivoted relative to each other, the position of the instrument drive unit 52 along the longitudinal axis of the holder 46 remains unchanged without the need to control the actuator 48c to compensate for movements of any of the first link 42b, second link 42c, third link 42a, and the holder 46.

The positioning of the actuator 48c in the first link 42b (e.g., proximate the first joint 44c) reduces the mass, inertia, and size of the tip of the robotic arm 40 (e.g., a region proximate the instrument drive unit 52 when the robotic arm 40 is in an extended condition), thereby enabling easier manipulation of the robotic arm 40. In particular, a lighter distal-most joint, enabled by virtue of the more proximal positioning of the actuator 48c, allows for less strain on the joints of the robotic arm 40 resulting in easier manipulation of the links due to a lesser degree of gravity compensation required during movement of the robotic arm 40 and its components.

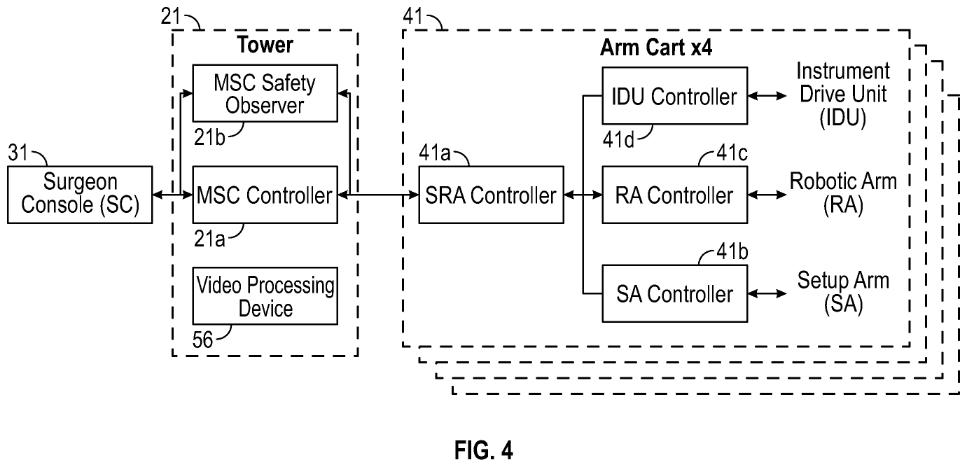

With reference to FIG. 4, each of the computers 21, 31, 41 of the surgical robotic system 10 may include a plurality of controllers, which may be embodied in hardware and/or software. The computer 21 of the control tower 20 includes a controller 21a and safety observer 21b. The controller 21a receives data from the computer 31 of the surgical console 30 about the current position and/or orientation of the handle controllers 38a and 38b and the state of the foot pedals 36 and other buttons. The controller 21a processes these input positions to determine desired drive commands for each joint of the robotic arm 40 and/or the instrument drive unit 52 and communicates these to the computer 41 of the robotic arm 40. The controller 21a also receives back the actual joint angles and uses this information to determine force feedback commands that are transmitted back to the computer 31 of the surgical console 30 to provide haptic feedback through the handle controllers 38a and 38b. The safety observer 21b performs validity checks on the data going into and out of the controller 21a and notifies a system fault handler if errors in the data transmission are detected to place the computer 21 and/or the surgical robotic system 10 into a safe state.

The computer 41 includes a plurality of controllers, namely, a main cart controller 41a, a setup arm controller 41b, a robotic arm controller 41c, and an instrument drive unit (IDU) controller 41d. The main cart controller 41a receives and processes joint commands from the controller 21a of the computer 21 and communicates them to the setup arm controller 41b, the robotic arm controller 41c, and the IDU controller 41d. The main cart controller 41a also manages instrument exchanges and the overall state of the movable cart 60, the robotic arm 40, and the instrument drive unit 52. The main cart controller 41a also communicates actual joint angles back to the controller 21a.

The setup arm controller 41b controls each of joints 63a and 63b, and the rotatable base 64 of the setup arm 62 and calculates desired motor movement commands (e.g., motor torque) for the pitch axis and controls the brakes. The robotic arm controller 41c controls each joint 44a and 44b of the robotic arm 40 and calculates desired motor torques required for gravity compensation, friction compensation, and closed loop position control of the robotic arm 40. The robotic arm controller 41c calculates a movement command based on the calculated torque. The calculated motor commands are then communicated to one or more of the actuators 48a and 48b in the robotic arm 40. The actual joint positions are then transmitted by the actuators 48a and 48b back to the robotic arm controller 41c.

The IDU controller 41d receives desired joint angles for the surgical instrument 50, such as wrist and jaw angles, and computes desired currents for the motors in the instrument drive unit 52. The IDU controller 41d calculates actual angles based on the motor positions and transmits the actual angles back to the main cart controller 41a.

The robotic arm 40 is controlled as follows. Initially, a pose of the handle controller controlling the robotic arm 40, e.g., the handle controller 38a, is transformed into a desired pose of the robotic arm 40 through a hand eye transform function executed by the controller 21a. The hand eye function, as well as other functions described herein, is/are embodied in software executable by the controller 21a or any other suitable controller described herein. The pose of one of the handle controller 38a may be embodied as a coordinate position and role-pitch-yaw (“RPY”) orientation relative to a coordinate reference frame, which is fixed to the surgical console 30. The desired pose of the surgical instrument 50 is relative to a fixed frame on the robotic arm 40. The pose of the handle controller 38a is then scaled by a scaling function executed by the controller 21a. In embodiments, the coordinate position is scaled down and the orientation is scaled up by the scaling function. In addition, the controller 21a also executes a clutching function, which disengages the handle controller 38a from the robotic arm 40. In particular, the controller 21a stops transmitting movement commands from the handle controller 38a to the robotic arm 40 if certain movement limits or other thresholds are exceeded and in essence acts like a virtual clutch mechanism, e.g., limits mechanical input from effecting mechanical output.

The desired pose of the robotic arm 40 is based on the pose of the handle controller 38a and is then passed by an inverse kinematics function executed by the controller 21a. The inverse kinematics function calculates angles for the joints 44a, 44b, 44c of the robotic arm 40 that achieve the scaled and adjusted pose input by the handle controller 38a. The calculated angles are then passed to the robotic arm controller 41c, which includes a joint axis controller having a proportional-derivative (PD) controller, the friction estimator module, the gravity compensator module, and a two-sided saturation block, which is configured to limit the commanded torque of the motors of the joints 44a, 44b, 44c.

It will be understood that various modifications may be made to the embodiments disclosed herein. In embodiments, the sensors may be disposed on any suitable portion of the robotic arm. Therefore, the above description should not be construed as limiting, but merely as exemplifications of various embodiments. Those skilled in the art will envision other modifications within the scope and spirit of the claims appended thereto.

It should be understood that various aspects disclosed herein may be combined in different combinations than the combinations specifically presented in the description and accompanying drawings. It should also be understood that, depending on the example, certain acts or events of any of the processes or methods described herein may be performed in a different sequence, may be added, merged, or left out altogether (e.g., all described acts or events may not be necessary to carry out the techniques). In addition, while certain aspects of this disclosure are described as being performed by a single module or unit for purposes of clarity, it should be understood that the techniques of this disclosure may be performed by a combination of units or modules associated with, for example, a medical device.

In one or more examples, the described techniques may be implemented in hardware, software, firmware, or any combination thereof. If implemented in software, the functions may be stored as one or more instructions or code on a computer-readable medium and executed by a hardware-based processing unit. Computer-readable media may include non-transitory computer-readable media, which corresponds to a tangible medium such as data storage media (e.g., RAM, ROM, EEPROM, flash memory, or any other medium that can be used to store desired program code in the form of instructions or data structures and that can be accessed by a computer).

Instructions may be executed by one or more processors, such as one or more digital signal processors (DSPs), general purpose microprocessors, application specific integrated circuits (ASICs), field programmable logic arrays (FPGAs), or other equivalent integrated or discrete logic circuitry. Accordingly, the term “processor” as used herein may refer to any of the foregoing structure or any other physical structure suitable for implementation of the described techniques. Also, the techniques could be fully implemented in one or more circuits or logic elements.

What is claimed is:

1. A surgical robotic system comprising:
a movable cart; and
a robotic arm operably coupled to the movable cart, the robotic arm including:
a first link pivotally coupled to a second link at a first joint and including an actuator, the second link including a first belt operably coupled to the actuator;
a holder pivotally coupled to the second link at a second joint, the holder including a first pulley, a second pulley, and a second belt coupled to the first pulley and the second pulley; and
an instrument drive unit operably coupled to the second belt and configured to move linearly along a longitudinal axis of the holder.

2. The surgical robotic system according to claim 1, wherein rotation of the actuator in a first direction causes the instrument drive unit to move along the longitudinal axis of the holder in a first linear direction and wherein rotation of the actuator in a second direction causes the instrument drive unit to move along the longitudinal axis of the holder in a second linear direction opposite the first linear direction.

3. The surgical robotic system according to claim 1, wherein the instrument drive unit is removably coupled to the second belt.

4. The surgical robotic system according to claim 1, further comprising a surgical instrument operably coupled to the instrument drive unit.

5. The surgical robotic system according to claim 1, wherein the actuator is integrated into the first link and is configured to produce an output oriented coaxially with the first joint.

6. The surgical robotic system according to claim 1, wherein the first pulley is coupled to the first belt at the second joint.

7. The surgical robotic system according to claim 1, further comprising a third link pivotally coupled to the first link at a third joint.

8. The surgical robotic system according to claim 7, further comprising a second actuator at the third joint, wherein the first link further includes a third belt operably coupled to the second actuator, and wherein actuation of the second actuator causes the first link to pivot relative to the third link and the second link.

9. The surgical robotic system according to claim 1, wherein an angle between the holder and the first link does not change upon movement of the second link relative to the first link.

10. A robotic arm for a surgical robotic system, the robotic arm comprising:
a first link pivotally coupled to a second link at a first joint and including an actuator, the second link including a first belt operably coupled to the actuator;
a holder pivotally coupled to the second link at a second joint, the holder including a first pulley, a second pulley, and a second belt coupled to the first pulley and the second pulley; and
an instrument drive unit operably coupled to the second belt and configured to move linearly along a longitudinal axis of the holder.

11. The robotic arm according to claim 10, wherein rotation of the actuator in a first direction causes the instrument drive unit to move along the longitudinal axis of the holder in a first linear direction and wherein rotation of the actuator in a second direction causes the instrument drive unit to move along the longitudinal axis of the holder in a second linear direction opposite the first linear direction.

12. The robotic arm according to claim 10, wherein the instrument drive unit is removably coupled to the second belt.

13. The robotic arm according to claim 10, further comprising a surgical instrument operably coupled to the instrument drive unit.

14. The robotic arm according to claim 10, wherein the actuator is integrated into the first link and is configured to produce an output oriented coaxially with the first joint.

15. The robotic arm according to claim 10, wherein the first pulley is coupled to the first belt at the second joint.

16. The robotic arm according to claim 10, further comprising a third link pivotally coupled to the first link at a third joint.

17. The robotic arm according to claim 16, further comprising a second actuator disposed at the third joint.

18. The robotic arm according to claim 17, wherein the first link further includes a third belt operably coupled to the second actuator.

19. The robotic arm according to claim 17, wherein actuation of the second actuator causes the first link to pivot relative to the third link and the second link.

20. The robotic arm according to claim 10, wherein an angle between the holder and the first link does not change upon movement of the second link relative to the first link.
